(19) United States
(12) Patent Application Publication
(10) Pub. No.: US 2003/0109855 A1
(43) Pub. Date: Jun. 12, 2003

(54) Method, a device, and a system for organ reconditioning and a device for preserving an internal body organ

(76) Inventors: Jan Otto Solem, Stetten (CH); Ronny Gustafsson, Lund (SE); Richard Ingemansson, Lund (SE)

Correspondence Address: BROWDY AND NEIMARK, P.L.L.C., 624 Ninth Street, N.W., Washington, DC, 20001, US

(21) Appl. No.: 10/259,822

(22) Filed: Sep. 30, 2002

(30) Foreign Application Priority Data
Sep. 28, 2001 (SE) 0103222-6

(51) Int. Cl. A61M 1/00

(52) U.S. Cl. 604/540; 604/313

(57) ABSTRACT

A device for reconditioning an internal body organ having or risking a functional failure or impairment associated with a fluid collection therein comprises a tube having a proximal end adapted for connection to a vacuum source, and a distal end portion having a plurality of openings. A chamber surrounds the distal end portion of the tube and the openings therein. A flexible material occupies said chamber and forms fluid connections between a selected part of an external surface of the chamber and the openings of the distal end portion of the tube. The selected part of the external surface of the chamber is adapted for contacting an external or internal surface portion of the internal body organ. Thereby, interstitial fluid of the internal body organ adjoining said selected part of the external surface of the chamber is sucked off from the internal body organ.

### TECHNICAL FIELD OF THE INVENTION

The present invention generally is related to reconditioning of internal body organs and more precisely is related to a device, a system and a method for reconditioning of an internal body organ having or risking a functional failure or impairment associated with a fluid collection therein. The present invention further relates to a device for preserving a body organ for transport purposes.

### BACKGROUND OF THE INVENTION

Functional failure or impairment of organ parenchyma is often caused by or associated with an increase of fluid, i.e. fluid collection which may cause a swelling, in the tissues (oedema). The fluid collection may cause a swelling, which in turn may result in a functional failure or impairment of an organ. However, a functional failure of an organ due to other causes may result in a fluid collection in the organ. The diseases causing these conditions may be very different. Infarctions, ischemia, and trauma may cause a situation where an increased amount of fluids leads to mal- or dysfunction of such internal body organs as the heart, the lungs, the kidney, the liver, the urinary tract, the guts, and the brain.

A heart infarction is caused by occlusions or blocks in the arteries of the heart. As a result of the block in the artery, a part of the heart will be deprived from nutrition and the heart muscle cells will die and go into necrosis. As a result of this process, water and other tissue fluids are accumulated in the diseased area of the organ. The increase of fluid in the organ tissue makes the diseased area or the whole organ stiff and a proper action of the heart muscle is inhibited.

Another situation when excessive fluid is present in the heart muscle is during the post cardiotomy syndrome, i.e. after extensive heart surgery when extra-corporeal circulation has been used and the heart has been arrested for a prolonged period of time.

A lung disease may also be impaired by excessive fluid in cases of infection, heart failure and shock. For shocked lungs, often called ARDS (adult respiratory distress syndrome), a major contribution to the failing function of the lungs is excessive fluid in the lung tissue.

The brain is an organ utterly sensitive to oedema and swelling. Since the skull represents a maximum volume, a swelling of the brain causes an immediate increase in the intracranial pressure. At a certain swelling status of the brain, the passage of blood and cerebral fluid is completely stopped through the foramen magnum, i.e. the opening into the skull.

Also, during organ preservation for transplant purposes oedema is a major concern, since accumulated fluid caused by ischemia and perfusion solutions inhibit good function immediately after transplantation and for the first post-operative period.

The normal transportation of fluids away from internal body organs is through the vascular system, basically through veins and the lymphatic system. Such fluid transportation through the vascular system is slow since fluids first have to be moved from the tissue between the organ cells into the vascular system along minimal gradients of osmotic pressure and capillary pressure.

Until now the single technique used for obtaining a faster reconditioning of a swollen internal body organ is general administrating of diuretics, a therapy that takes time and has disadvantages like electrolyte imbalance and dehydration of other parts of the body.

### SUMMARY OF THE INVENTION

Due to the above-mentioned problems, an object of the present invention is to provide a method, a device, and a system for reconditioning of an internal body organ having, or risking a functional failure or impairment associated with a fluid collection therein.

This object is attained by means of a device and a method according to the appended independent claims. Preferred embodiments of the invention are stated in the dependent claims.

According to the invention there is provided a device for reconditioning of an internal body organ having, or risking a functional failure associated with a fluid collection therein, comprising: a tube having a proximal end adapted for connection to a vacuum source, and a distal end portion having a plurality of openings; a chamber surrounding the distal end portion of the tube and the openings therein; and a flexible material occupying said chamber and forming fluid connections between a selected part of an external surface of the chamber and the openings of the distal end portion of the tube, said selected part of the external surface of the chamber being adapted for contacting the internal body organ, whereby interstitial fluid of the internal body organ adjoining said selected part of the external surface of the chamber may be sucked off from the internal body organ.

Thus, the invention provides an increased fluid flow away from a swollen organ, thereby contributing to a faster reconditioning of the swollen organ.

According to another aspect of the invention, there is provided a device for reconditioning of an internal body organ having, or risking a functional failure or impairment associated with a fluid collection therein, said device comprising: an organ contacting surface, which is adapted to contact the internal body organ, said organ contacting surface having, at least during the use of the device, pores allowing interstitial fluids to flow from the internal body organ through the surface; and a draining element adapted to apply a negative pressure at the organ contacting surface and adapted to lead said interstitial fluids away from the internal body organ at said organ contacting surface via said pores.

According to the invention, there is also provided a system for reconditioning an internal body organ having, or risking a functional failure or impairment associated with a fluid collection therein, said system comprising

(i) an organ contacting device, said organ contacting device including:

an organ contacting surface, which is adapted to contact the internal body organ, said organ contacting surface having pores allowing interstitial fluids to flow through the surface; and

a draining element adapted to apply a negative pressure at the organ contacting surface and adapted to lead sucked off fluids away from the organ at said organ contacting surface; and

(ii) a negative pressure source for connection to the draining element and for creation of the negative pressure at the organ contacting surface.

Further, a method according to the invention for reconditioning an internal body organ having, or risking a functional failure associated with a fluid collection therein, comprises the steps of providing a tube, which has a proximal end and a distal end portion having a plurality of openings, a chamber surrounding the distal end portion of the tube and the openings therein, and a flexible material in said chamber and forming fluid connections to the openings of the distal end portion of the tube; contacting the internal body organ by said flexible material; and connecting the proximal end of the tube to a vacuum source, whereby interstitial fluid of the internal body organ adjoining said selected part of the external surface of the chamber is sucked off from the internal body organ.

According to another aspect of the invention, a method for reconditioning of an internal body organ having, or risking a functional failure or impairment associated with a fluid collection therein comprises the steps of contacting the internal body organ with a suction device, and creating a negative pressure in a contact area between the internal body organ and the suction device, whereby interstitial fluid is sucked off from the internal body organ.

According to a further aspect of the invention, a method for reconditioning of an internal body organ having, or risking a functional failure or impairment associated with a fluid collection therein comprises the step of sucking off interstitial fluid from an external surface or from the interior of the internal body organ.

According to yet another aspect of the invention, a method for reconditioning of an internal body organ having, or risking a functional failure or impairment associated with a fluid collection therein comprises the step of increasing fluid flow away from the internal body organ by applying a negative pressure to the internal body organ.

Accordingly, the present invention uses suction or gentle vacuum on the organ surface and thereby directs the fluid flow in a direction opposite to the normal fluid flow away from the organ, i.e. out of the organ through the outer layer of the organ surfaces, e.g. the epicardium, and the pleura of the lung. In comparison to the use of diuretics, a direct removal of excessive fluids from the organ surface will create neither electrolyte imbalance nor dehydration of other parts of the body. Further, the direct removal via the organ's surface will work fast, so that excessive fluid can be removed in an early stage of a disease. Thereby, a chronic state with cell inflammation and scar tissue formation may be avoided.

Fluid collection in an organ implies that fluid is accumulated in cells and between cells of the organ. When a negative pressure is applied to the organ in accordance with the invention, interstitial fluid, i.e. fluid between the cells, will be sucked off from the organ. However, the sucking off of interstitial fluid will result in intracellular fluid, i.e. fluid within the cells, moving out from the cells by osmotic pressure, whereby this fluid may also be sucked off from the organ. The interstitial fluid is a liquid, but gases may accompany the fluid when it is sucked off. Further, the sucked off fluid may carry toxins and other inflammatory substances as well as free radicals away from the organ. The term “interstitial fluid” does not include blood or fluids in a body cavity outside organs.

The term “reconditioning” should be interpreted in a broad sense. In the present application, “reconditioning” will include not only the action of removing an existing fluid collection of an internal body organ but also the action of preventing an expected fluid collection of an internal body organ.

The feature that the chamber “surrounds” the distal end portion of the tube and the openings thereof implies that at least the openings will be covered by the chamber.

U.S. Pat. No. 5,636,643 discloses a device for a completely different purpose than reconditioning of internal body organs. The device according to U.S. Pat. No. 5,636,643 is used for treatment of wounds. The device comprises a tube for providing a suction, a wound screen for application on the wound and around a distal end of the tube. When the device is arranged on a wound and a negative pressure is applied to the wound, the healing of the wound will be stimulated and accelerated.

Further, U.S. Pat. No. 6,015,378 discloses another device that also somewhat resembles the device according to the invention. The device according to U.S. Pat. No. 6,015,378 is used for the purpose of stabilizing a tissue area. Thus, the device comprises a suction device and suction openings at a distal end of the suction device. When a negative pressure is applied to the suction device, a body tissue in the vicinity of suction openings will be immobilized by a suction force binding it to the suction opening.

According to an aspect of the invention, a special feature of the device and its surface towards the organ to be reconditioned is the properties of the surface to prohibit, or at least substantially reducing, stimulation of granulation tissue. Such granulation tissue is a specific tissue for healing of wounds and tissue damages, especially such damage that is caused by infections and burns. The application of an organ contacting surface in contact with an internal body organ may stimulate formation of unwanted granulation tissue, if the surface is not designed to prohibit this reaction. Granulation tissue contains tissue like capillaries, inflammatory cells and fibrocytes or fibroblasts. Such cells may originate from the blood or the tissue itself. Such wound healing reaction is unwanted during reconditioning by means of the device and the device is designed to avoid such reactions.

One way to avoid granulation at the surface is to choose material and/or porosity of the device surface such that the granulation tissue stimulation is avoided. Thus, the organ contacting surface may have pores of a size sufficiently small to prevent stimulation of granulation tissue formation. The pores of the organ contacting surface are preferably of a size in the interval 2-300 μm. It is even more preferred that the size of the pores is in the interval 2-4 μm. In these intervals, the pores will be sufficiently big to allow interstitial fluids to flow through the surface, while the pores still are sufficiently small to prevent stimulation of granulation tissue formation. Further, the pores will also be smaller than the size of the blood cells to prohibit exsanguination of the patient or sucking any parenchymal cells.

In other words, the pores of the flexible material may typically be of a size in the interval 2-300 μm at the selected part of the external surface of the chamber, since small pores will not stimulate granulation tissue generation as much as large pores.

The pores of the organ contacting surface may initially be clogged. Then, a negative pressure applied by the draining element will not affect the surroundings of the organ contacting surface but may instead be used for compressing the part of the device carrying the organ contacting surface, which is to be introduced into the body. Preferably, the pores are clogged by a resorbable material. Thus, the material clogging the pores may be resorbed when the organ contacting surface is introduced into the body. Then, the pores are cleared so that interstitial fluid may flow through the organ contacting surface when a negative pressure is applied by the draining element.

Also, since the surface prohibits, or at least substantially reduces, granulation tissue stimulation, the device may easily be replaced as it will not attach to the organ surface. Further, the device may be arranged for creating an overpressure to the body organ surface. The creation of an overpressure may be used for detaching the device from the body organ surface when the device is to be replaced or retrieved. In particular, the overpressure may be used for breaking any granulation tissue formations. An occasional overpressure may also be used for breaking any granulation tissue formations during the application of the device in contact with the body organ surface. This overpressure may be applied periodically.

Alternatively, the selected part of the external surface may comprise a material, drug or substance, that prohibits, or at least substantially reduces, adhesion and granulation tissue formation. Thus, an adhesion of the device to the body organ is prohibited.

Further, the method according to the invention may comprise the step of prohibiting, or at least substantially reducing, stimulation of granulation tissue at a contact area of the flexible material to the surface of the body organ, and/or the step of prohibiting, or at least substantially reducing, stimulation of adhesion of the contact area to the surface of the body organ.

Preferably, at least part of a peripheral surface of a porous body forms the organ contacting surface. Thus, the organ contacting surface provides an interface between a surface of a body organ and a porous body. The porous body may enable the application of a negative pressure at the organ contacting surface by coupling the draining element to the organ contacting surface. Peripheral parts of the porous body which do not form the organ contacting surface may be covered by a film. This may prohibit fluid flow into the porous body through other parts than the organ contacting surface.

The porous body may be flexible. This may be advantageous in that the porous body may then be shrunk by an applied negative pressure inside it, which will facilitate introduction of the porous body into a human body.

Preferably, a distal end of the draining element is arranged inside the porous body. Thus, the negative pressure may be applied through the porous body and sucked off fluids will be led through the porous body from the organ contacting surface to the draining element. The draining element may comprise a tube for leading away fluids. Preferably, the distal end of the draining element comprises at least one suction opening in the tube. Through this suction opening fluids may be led away from the body organ by means of the applied negative pressure. In particular, the draining element may comprise a plurality of suction openings. These suction openings are preferably distributed in relation to the organ contacting surface. Thus, the negative pressure applied to the organ contacting surface may be uniformly distributed over the organ contacting surface. Preferably, the distal end of the draining element extends in the porous body substantially in parallel to the organ contacting surface. As a result, suction openings distributed over the length of the distal end will be distributed in relation to the organ contacting surface. The draining element may also be connectable to a vacuum source for providing the negative pressure to the organ contacting surface.

In a first embodiment, the selected part of the external surface of the chamber, or in other words the organ contacting surface, is adapted for contacting an external surface portion of the body organ. Thus, the body organ may be treated, while no penetration of the organ tissue is needed. According to this embodiment, a negative pressure may be applied to an external surface of the body organ for sucking off interstitial fluid.

Preferably, the non-selected part of the external surface of the chamber is impermeable to fluids. The non-selected part of the external surface of the chamber may also be impermeable to gases. Further, the non-selected part of the external surface of the chamber may comprise a film.

In the first embodiment, the device according to the invention may have a flat chamber with two opposing sides. Then, the selected part of the external surface of the chamber may be positioned on only one of the two opposing sides.

This embodiment is well suited for attaching the device to such organs as the heart, the lungs, the kidney, the liver, and the guts. The selected part of the external surface of the chamber may be enclosed by a sealing ring, thereby ensuring a tight attachment. Preferably, the sealing ring comprises a sealing lip.

The device may also comprise means for creating a negative fixation pressure between the sealing ring and a surface of a body organ, whereby the sealing ring may be fixated to the surface of the body organ. For example, the sealing ring may have a U-shaped cross-section. If a negative pressure is applied, the U-shaped sealing ring will be fixated to a surface below the opening of the U-shape. Alternatively, the sealing ring may comprise several suction cups, which may be fixated to the surface of the body organ.

Also, the sealing ring is preferably impermeable to fluids and it may further also be impermeable to gases. This enables a tight fixation of the device to the body organ.

Also, the chamber may be divided into compartments. Each such compartment may comprise a portion of the selected part of the external surface of the chamber. In other words, the porous body may be divided into compartments, wherein part of a peripheral surface of each compartment form separate portions of the organ contacting surface. In this case, the tube may have a separate lumen for each compartment, or each compartment may have a separate tube for connection to a vacuum source. Thus, the draining element may have a separate lumen for each compartment, or may comprise several tubes, wherein each tube comprises a distal end arranged in one of the compartments. This configuration enables a varied suction across the surface of a treated organ.

Further, each compartment may have a sealing ring, preferably comprising a sealing lip, enclosing its portion of the selected part of the external surface of the chamber. In other words, a sealing ring may enclose each portion of the organ contacting surface. Also, the sealing ring of each compartment may comprise means for creating a negative pressure between the sealing ring and a surface of a body organ, whereby the sealing ring may be fixated to the surface of the body organ. The sealing ring of each compartments may each comprise a sealing lip.

The selected part of the external surface of the chamber or the organ contacting surface is suitably covered by a perforated film. Then, the flexible material or the organ contacting surface will not be in direct contact with the surface of the organ, but still fluid connections could be formed.

The porous body may comprise a spongy material. Alternatively, the flexible material or, in other words, the porous body preferably comprises a web, which could comprise e.g. a synthetic tissue selected among the group consisting of polyester, polydioxanon, polyhexafluoropropylen-VDF, polyglyconat, polyglycolic acid, polyglactin, and silicon. The web may also comprise a metal material, such as Nitinol. Alternatively, the web may comprise a combination of a metal and a synthetic material. These configurations of the flexible material or the porous body will enable fluid flow through the material. Also, the flexible material may be a resorbable material, thus enabling the flexible material to be left in the body after the treatment.

According to a further alternative, the web comprises at least one layer of a net. The net may be fine-meshed, thus forming pores of the web. The web might comprise several layers of net on top of each other creating a multi-layer net. The mesh size of the layers may then differ at the various levels allowing individual design for different organs and fine-tuning function.

Further, the selected part of the external surface of the chamber or the organ contacting surface preferably restrains shrinking due to a negative pressure in the tube creating a suction. This could be accomplished by a sealing ring sufficiently rigid to maintain its form under the negative pressure. The sealing ring will then prohibit the selected part of the external surface from shrinking. Such shrinking and subsequent stiffness of the flexible material would be of definitive disadvantage when the device would be used e.g. on a beating heart. Also, the material of the distal end portion of the tube may restrain shrinking due to the negative pressure.

The resistance towards shrinking implies that the area of the selected part of the external surface may be held constant. Thus, the flexible material may have an essentially stable form in a plane of the selected part of the external surface, while being pliable such that the selected part of the external surface may be bent and formed to come in contact with a curved surface of the body organ. During this bending the area of the selected part of the external surface is not changed.

In a second embodiment, the selected part of the external surface of the chamber, or in other words the organ contacting surface, is adapted for contacting an interior portion of the body organ. Then, the selected part of the external surface may be brought in close proximity to a fluid collection in the body organ. According to this embodiment, a negative pressure may be applied to the interior of the body organ.

In this second embodiment, the device according to the invention may have a chamber, which is substantially cylindrical. Then, the selected part of the external surface of the chamber includes the peripheral part of the substantially cylindrical chamber, preferably the total external surface of the chamber. In other words, the porous body may be substantially cylindrical and at least part of the peripheral surface of the substantially cylindrical porous body may form the organ contacting surface.

Preferably, the device further comprises a delivering cannula, into which the chamber or porous body may be inserted in a compressed state. This enables a simple introduction of the chamber into the body and to an organ to be reconditioned.

This embodiment is preferred for treatment of a swelling of the brain. By inserting a device according to this second embodiment of the invention into the brain tissue, oedemas may be treated before the above-described disastrous development occurs, such oedemas being the main cause of death after head injury, vascular disasters in the brain and after brain surgery. The device may be inserted through the skull bone where a screw will tighten the insertion hole and prohibit leakage, or through the vascular system, either through a vein or through an artery. The device may also be inserted into the skull under the bone but outside of the brain tissue, sucking on the brain surface, inside or outside the dura mater.

Such treatment inside the organ parenchyma (i.e. between the organ cells and not on an organ surface) may also be used in other organs, such as the heart or the kidneys. In such treatment inside the organ parenchyma, the pores of the selected surface is preferably selected in the interval of 2-4 μm, i.e. smaller than the size of the blood cells to prohibit exsanguination of the patient or sucking any parenchymal cells.

Since the processes of fluid increase in the heart may be very fast, the invention also provides for a monitoring of the function and the recovery of the heart muscle cells. If the monitoring discloses a need for immediate direct therapy, like electrical stimulation or D.C. shock, the device according to the invention may permit such action. The device may also permit localized distribution of drugs in the diseased tissue as well as direct pacemaker stimulation of the heart surface. The placement of the device on the heart surface may be done directly when the chest is opened, or it may be applied to the heart surface by percutaneous direct puncture into the pericardial space. The system may also be inserted into the organ parenchyma by means of a percutaneous puncture technique through the skin directly into the pericardium or through the vascular system by veins or arteries.

Myocardial infarction is an event caused by sudden blocks in the arteries supporting a certain area of the muscle. The blocks are caused by blood clots, so called thromboses. The acute ischemia that occur causes death of heart muscle cells in the area and swelling by fluid collection caused by necrosis of the cells and oedema. The presented device is ideal for treatment of such areas of dead swollen muscle cells. If, however the block in the artery is opened by means of drugs, balloon dilatation or acute bypass surgery such cell death may be omitted or limited. However, immediately after restoring blood flow in the previously blocked areas the recurring blood flow causes so-called reperfusion damage, i.e an oedema in the ischemic area and also in the border zones of the ischemic area. Such oedema impair the myocardial contraction, but the presented device may cure such impairment instantly. If reopening the artery is unsuccessful cell death will occur and a border zone swelling will develop impairing also the surviving cells in the border zone. By applying the device in the swollen border zone the global function of the heart will improve as well as survival after the myocardial infarction. Ischemic areas next to necrotic myocardial cells are called stunned or hibernating myocardial tissue suffering from deficient blood support, these areas are also the areas causing the alarming and frightening pain of the unstable angina pectoris. The presented invention may cure such starvation of blood supply by means of another feature, that is its ability to increase blood flow in the small vessels called capillaries and arterioles in the stunned or hibernating areas. By clearing the area of superfluous fluid and by sucking in the venous end of the capillaries a passive blood supply may be created from the adjacent healthy areas into the starved areas treated by the presented device.

By altering the suction modus in the device, efficiency of the device may be increased and thereby also an enhancement of the microcirculation may be created. Such altering typically would include a fine tuning of the suction force by tuning the applied negative pressure to an interval between 25 mmHg to 125 mmHg and also making the variation of suction force cyclic, e.g. increasing and decreasing suction at an interval of 1-3 minutes. Another feature of the present invention is that, in addition or instead of the 1-3 minutes variation of suction, a much faster variation of the suction force may be triggered by the detected electrocardiogram (ECG) to determine an optimal period to the variation in each cardiac cycle. Thus, the variation according to the ECG may be superposed on the 1-3 minutes variation, or alternatively control the whole variation. Typically the ECG triggered variation would peak the suction in the diastole of the heart cycle when the blood support of the left heart side is at its maximum. However the systole of the heart cycle may also be chosen for increasing the microcirculation in other areas of the heart, for instance the right side of the heart. It is obvious from what is mentioned above that such increase in the microcirculation in the ischemic areas causing pain may cure the very often intractable chest pain of unstable angina pectoris. It is also obvious that such an increase in microcirculation will salvage and supply heart muscle cells during a period of regeneration of blood vessels to the damaged area and thereby permit a permanent salvage of that heart muscle area.

Preferably, the device may further comprise an electrical current conducting means adapted for contacting the body organ. The electrical current conducting means may comprise at least one electrode. For instance, the electrical current conducting means may be adapted for detecting an electrical current. Then, the electrical current conducting means may be adapted for detecting an electrical current in the body organ. This detection may be used for monitoring ECG signals of a heart.

The electrical current conducting means may also or alternatively be adapted for detecting an electrical current influenced by the fluid flow away from the body organ, e.g. by a change of conductivity. Thus, the flow of interstitial fluid sucked off from the body organ may be monitored. Monitoring the flow of interstitial fluid from the body organ gives a view of how fast the process of treating the body organ progresses. It may also indicate when the body organ needs no further treatment, i.e. when the flow is at a level corresponding to treatment of a healthy organ. Thus, a degree of vacuum in the vacuum source may be regulated in dependence of the monitored flow. Also, the vacuum source may be shut off when the monitored flow is at a normal level.

Further, the electrical current conducting means may be adapted for applying an electrical current to the body organ. In this case, the electrical current conducting means may comprise a net layer of metal for applying an electrical current to the body organ over a distributed surface. The net layer of metal may be arranged at the organ contacting surface. The electrical current conducting means may alternatively comprise a metal wire around the organ contacting surface for achieving a distributed application of an electrical current to the body organ. This may be used for electrical stimulation regulating the heart rhythm (pacemaker) or a D.C. shock to a heart, if this is needed during a treatment of the heart.

According to the invention, there is provided a device for preserving a body organ for transport purposes comprising: having a web inside; at least one tube having a proximal end adapted for connection to a vacuum source and a distal end portion having a plurality of openings; a flexible sealed bag surrounding the distal end portion of the tube and the openings therein; and a flexible material positioned on the inside of the flexible bag, the openings in the distal end of the tube being positioned within the web, which forms fluid connections between an external surface of a body organ placed in the bag and the openings of the distal end portion of the tube, whereby interstitial fluid of the body organ adjoining the web is sucked off from the body organ.

According to another aspect of the invention, there is provided a device for preserving a body organ for transport purposes, said device comprising: a sealable bag having an internal space for receiving a body organ; and a draining element adapted to apply a negative pressure to an external surface of the body organ and adapted to lead sucked off fluids away from the body organ.

The transportation and storage period for organs to be transplanted may be used for reconditioning by use of the present invention. By treating the whole organ or the organ surface with gentle vacuum during transportation and storage, and also after transplantation, excessive oedemas may be extracted and a superior function of vacuum treated organs will be ensured. Such treatment may be very advantageous for kidney, livers, lungs and hearts intended for transplantation. The device has a sealable bag, whereby a suction may be provided to an organ in the bag. The draining element need not be in actual contact with the organ in order to apply the negative pressure for sucking off fluid from the organ.

Preferably, the device for preserving a body organ for transport purposes further comprises an organ contacting surface located within the bag and adapted to contact the body organ. The organ contacting surface may be connected to the draining element for applying a negative pressure to the external surface of the body organ. The organ contacting surface may be used for applying the negative pressure to at least a part of the body organ and for forming fluid connections between the body organ and the draining element.

In the device for preserving a body organ for transport purposes, the organ contacting surface is preferably formed by a peripheral surface of a porous body. Further, the porous body may comprise a flexible material. Then, the porous body could conform to the shape of the organ in the bag. Also, the porous body may comprise a web.

The draining element of the device for preserving a body organ may comprise at least one tube and preferably comprises several tubes. At least one suction opening may be arranged at a distal end of each tube, which distal end may be arranged in the porous body. Thus, fluids may be sucked off from the body organ in the bag through the organ contacting surface, the porous body and the suction opening, whereby the fluids may be led away from the bag through the tube. Further, the draining element may be adapted for connection to a vacuum source for providing a negative pressure at the organ contacting surface.

Further, the device for preserving a body organ may be placed inside a cooling box or in a cooled environment to keep the organs at a lowered temperature. This enhances the preservation of the body organ.

### BRIEF DESCRIPTION OF THE DRAWINGS

Preferred embodiments of the invention will now be described referring to the appended drawings, wherein

FIGS. 1-3 are a top view, a side view and a bottom view of a first embodiment of a device according to the present invention.

FIG. 4 is a perspective view of a preferred embodiment of a sealing ring.

FIGS. 5-10 illustrate three further variants of a device according to the first embodiment of the present invention.

FIGS. 11-12 are a top view and a cross-sectional view of another variant of the device according to the first embodiment of the present invention.

FIGS. 13-14 are a top view and a cross-sectional view of still another variant of the device according to the first embodiment of the present invention.

FIGS. 15-16 illustrate the use of a device according to the present invention.

FIG. 17 illustrate two devices according to FIGS. 1-3 attached to the anterior wall of the heart and the backside of the heart.

FIGS. 18 and 19 illustrate the use of four devices according to FIGS. 13-14 for reconditioning of the lungs.

FIGS. 20-22 illustrate how a device according to FIGS. 1-3 may be minimized for insertion into a cannula.

FIGS. 23-25 illustrate a second embodiment of the device according to the present invention which are intended for insertion into the organ, i.e. an intraparenchymal device for insertion into the tissue of the organ.

FIGS. 26 and 27 are partial cross-sectional views of a skull and illustrate the use of the devices shown in FIGS. 23-25.

FIGS. 28-30 illustrate a third embodiment of a device according to the invention intended for preservation of a body organ for transplantation.

FIGS. 31-32 illustrate alternative embodiments of the sealing ring.

FIG. 33 illustrates a way of inserting the device according to FIGS. 1-3 into the body.

### DETAILED DESCRIPTION OF PREFERRED EMBODIMENTS

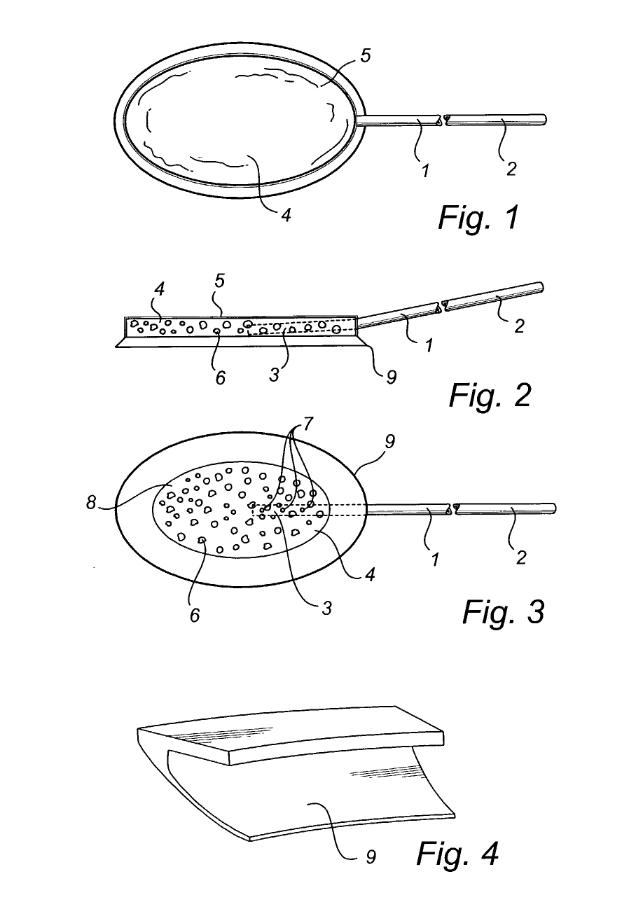

The device illustrated in FIGS. 1-3 constitutes a first embodiment comprising a draining element having a tube 1 with a proximal end 2 adapted for connection to a vacuum source (not shown), and a distal end portion 3 inserted into a flat chamber 4 having a flexible shape. A top side of the chamber 4 is covered by a thin film 5 which is impermeable to fluids and gases. The chamber 4 is filled by a flexible material 6 which may be a web, e.g. consisting of a synthetic tissue selected among the group consisting of polyester, polydioxanon (PDS), polyhexafluoropropylen-VDF (Pronova), polyglyconat (Maxon), polyglycolic acid (Dexon), polyglactin (Vicryl), and silicon. It is the flexible material 6 of the chamber 4 that makes the shape of the chamber 4 flexible. However, the flexible material 6 permit the chamber 4 to reduce its volume when a negative pressure is applied. The flexible shape of the chamber 4 implies that the chamber may be bent and flexed to adapt to the form of an organ surface. However, a surface of the chamber 4, which is to be in contact with the organ, is essentially form stable, i.e. it may be bent but the area of the surface may not be compressed.

The web might also be created by putting nets of the materials mentioned on top of each other creating a multi-layer net. However, only a few or even only one layer may be used. The size of the masks in the net may then differ at the various levels allowing individual design for different organs and fine tuning function. The web may also be made of metal in form of metal sheets with masks in one or multiple layers, or alternatively it may also be made of one or multiple thin threads of metal distributed in a predetermined manner or at random. Typically, one such metal would be Nitinol, i.e. an alloy of nickel and titanium with inherent shape memory function. However, a combination of one or more of the synthetic materials and metal may also be used.

The flexible material 6 comprises pores and openings. Therefore, gases and fluids may be transported through the flexible material 6. Preferably, the pores of the flexible material are of a size smaller than 300 μm, since this prohibits, or at least substantially reduces, stimulation of granulation tissue creation when the flexible material 6 comes in direct contact with an organ surface, as described above.

The draining element could alternatively be described as having a porous body and a tube, wherein a distal end portion of the tube is arranged within the porous body.

The distal end portion 3 of the tube 1 has at least one, but preferably a plurality of holes 7 or suction openings. If several holes 7 are arranged in the tube 1, it is not so critical if a hole 7 is clogged. The flexible material 6 in the chamber 4 forms fluid connections through its pores. Thus, fluid connections are formed between these holes 7 and a selected part 8 of an external surface of the chamber 4. The selected part 8 of the external surface of the chamber 4 may also be described as an organ contacting surface formed by a peripheral surface of the porous body. In FIG. 3 this selected part is illustrated as part of the bottom side of the chamber 4. The flexible material 6 may be exposed within the selected part 8 of the external surface of the bottom side of the chamber 4, or it may be covered by a perforated film.

The selected part 8 of the external surface of the chamber 4 is permeable to fluids, whereby the fluid connections could be formed. Also, the selected part 8 of the external surface may be seeded or covered with a material, drug or substance, that is prohibitive against adhesion and stimulation of granulation tissue. Such material or substance may be silver or carbon, but any other substance that is accepted by the human body and have the same effect might be used. Drugs with such effects are typically steroids, anti-inflammatory drugs like ibubrufen and similar drugs, and also cytostatic agents and cytotoxic agents.

The holes 7 are preferably formed on a side of the distal end portion 3 of the tube 1 proximal to the selected part 8 of the external surface of the chamber 4. The distal end of the tube 1 is preferably closed so that the vacuum source could create a suction through the holes 7. Preferably, the holes 7 in the tube 1 are distributed in relation to the selected part 8 of the external surface of the chamber 4 such that a substantially uniform negative pressure may be applied at the selected part 8 of the external surface. The distribution of the holes 7 may also be used for achieving a controlled variation of the negative pressure over different parts of the selected part 8 of the external surface.

The pores of the organ contacting surface or of the selected part 8 of the external surface may initially or temporarily be filled by a biodegradable or resorbable material. Alternatively, the selected part 8 of the external surface may be covered completely by a film of a resorbable material.

Thus, if a negative pressure is applied through the tube to the chamber or porous body, it will be compressed to a very small size, since air in the flexible material could be sucked away through the tube. This is possible since the surface of the chamber is tight as the pores of the surface are filled by a resorbable material. This may advantageously be used for insertion of the device into contact with the body organ. When the surface is arranged in contact with the body organ, the resorbable material will be resorbed or decomposed. With open pores the device will swell to its natural size, whereby fluid flow through the pores of the selected part 8 of the external surface is once again enabled. This implies that the device may be inserted into the body in a very small size and then suction of fluids through the device will automatically be enabled in the body. The resorbable material could be PDS (polydioxanon), Pronova (polyhexafluoropropylen-VDF), Maxon (polyglyconat), Dexon (polyglycolic acid), Vicryl (polyglactin), any kind of saccaride, or human albumin.

The selected part of the bottom surface of the chamber 4 is enclosed or surrounded by a sealing ring 9, which in a preferred embodiment comprises a sealing lip, illustrated in FIG. 4. The sealing lip has a slack extension towards the chamber 4. A negative pressure in the chamber 4 will support fixation of this extension to a surface under it.

When a negative pressure is applied to the chamber 4, the sealing ring 9 is sufficiently rigid to restrain shrinking of the selected part 8 of the external surface of the chamber 4. Thus, the application of a negative pressure will not cause shrinking of the body organ, which otherwise could affect the function of the body organ negatively.

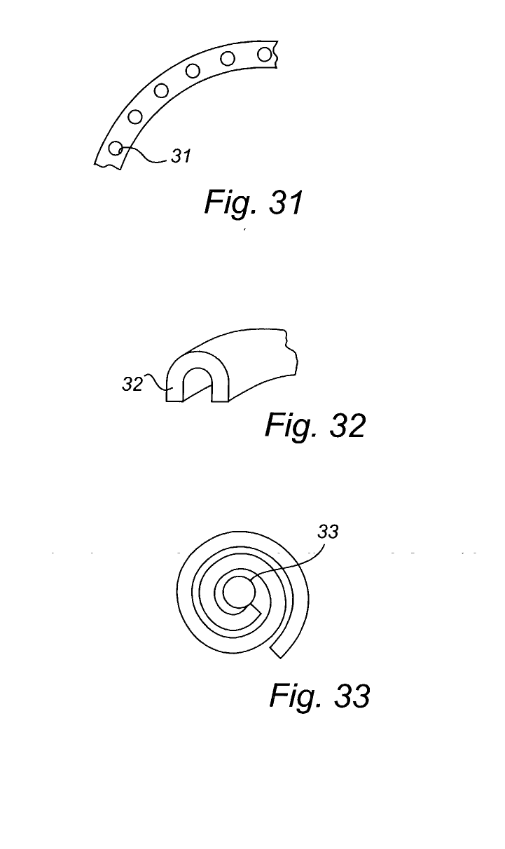

Referring to FIGS. 31-32, the sealing ring 9 could also be connected to a further vacuum source (not shown) for fixating the sealing ring 9 to a body organ by suction. Thus, the sealing ring 9, on its external surface which is to be in contact with the body organ, could e.g. comprise holes 31, suction cups, or have a U-shaped cross-section 32 with the opening towards the body organ when the device is applied in contact with the organ. This hole 31, suction cup or U-shaped cross-section 32 is then connected to the vacuum source for creating a negative pressure between the opening of the sealing ring 9 and the body organ and thus attaching the sealing ring 9 to the body organ.

The selected part 8 of the external surface of the chamber 4 is adapted for contacting an external surface portion of a body organ. When connecting the proximal end of the tube 1 to a vacuum source, the pressure within the chamber 4 could be decreased such that the chamber 4 will be tightly fixed against the external surface of the body organ by means of the sealing ring 9, while the flexible material 6 in the chamber 4 will prevent the chamber from deflating. The sealing ring 9 will thus be tightly fitted to the surface of the body organ. A closed space is defined for creating a negative pressure on the surface of the body organ. The pressure decrease will generate a suction effect on the external surface of the body organ inside the sealing ring 9, whereby interstitial fluid of the body organ adjoining said selected part 8 of the external surface of the chamber 4 is sucked off from the body organ.

The proximal end 2 of the tube 1 will be connected to the vacuum source outside the body. However, the vacuum source need not be connected to the proximal end of the tube 1 but could also be connected to a proximal connecting portion outside the body. The proximal connecting portion need not be formed in the end of the tube; instead it may e.g. be in a proximal branch of the tube.

The fluid extracted from the body organ will be separated into a receptacle (not shown) connected into and interrupting the tube 1 between its distal and proximal ends.

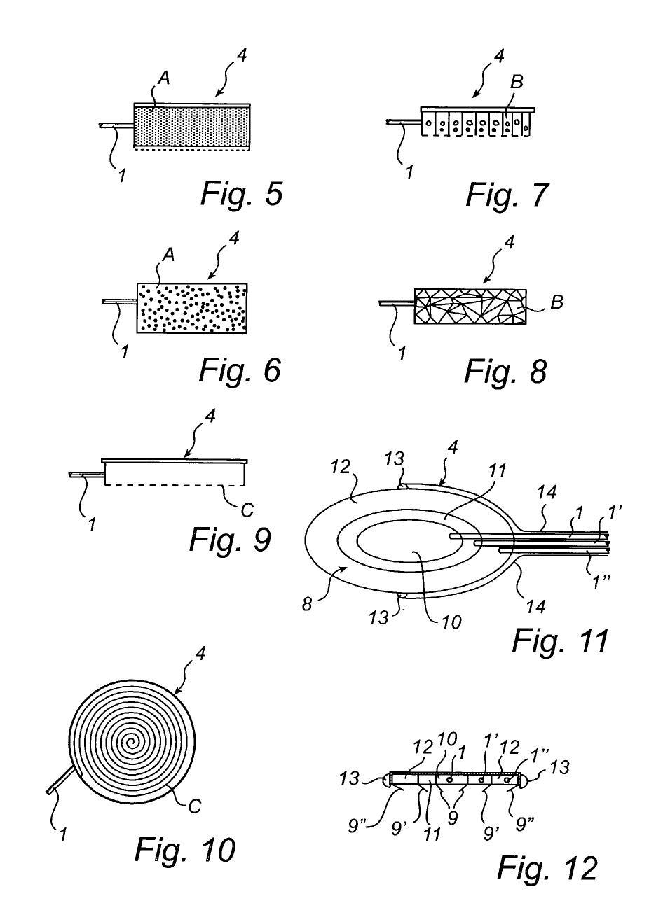

Alternatively, the flexible material 6 may consist of any material separating the film 5 forming the top side of the chamber 4 and a perforated film forming the bottom side, or at least the selected part 8 of the bottom side of the chamber 4. Examples of such separating material are shown in FIGS. 5-10 as a plurality of tabs A, a honeycomb structure B and a spiral of a plastic tubing C, respectively. In these variants, the flexible material 6 is a structure with holes or openings. These holes or openings of the flexible material 6 will then form the fluid connections between holes in the perforated film and the suction openings 7.

FIGS. 11-12 illustrate a variant of the first embodiment of the device illustrated in FIGS. 1-3. However, the chamber 4 is separated into three different compartments 10, 11 and 12, each one of the compartments 10-12 comprising a portion of the selected part 8 of the external surface of the chamber 4. Further, each compartment has a separate tube 1, 1′, 1″ leading to a vacuum source (not shown). Alternatively, there may be a single tube having a separate lumen or opening for each compartment. Each one of the compartments 10-12 may have a sealing ring 9, 9′ and 9″ enclosing its portion of the selected part 8 of the external surface of the chamber 4.

Obviously, by separating the chamber 4 into several compartments it is possible to vary the pressure across the surface of the body organ covered by the selected part 8 of the external surface of the chamber 4.

Each one of the sealing rings 9, 9′ and 9″ may be such a sealing lip as illustrated in FIG. 4. These lips may even extend to the top side film 5 and thus separate the compartments 10-12.

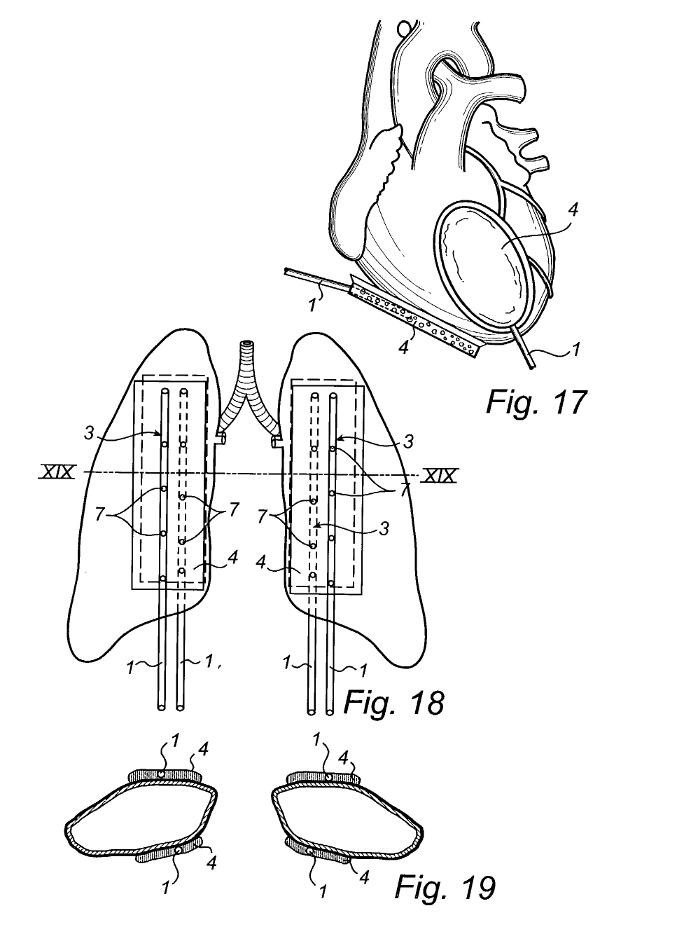

Metal contact points in the form of electrodes 13 are shown in FIGS. 11-12 as are wires 14. The wires 14 may connect the electrodes 13 to a detecting unit for recording ECG signals or to a pacemaker for stimulating purposes, when the device is fixed on a heart, as shown in FIG. 17. As stated above, the device according to the invention may permit immediate direct therapy, like electrical stimulation or D.C. shock, if the monitoring discloses a need for such action.

The electrodes 13 may be used for detecting an electrical current. This may be accomplished by having two spaced apart electrodes 13, which detect a current between them. The detection may be used for monitoring a current in the body organ, such as ECG signals. Through this detection the condition of the organ that is being treated may be monitored.

The detection may also be used for monitoring the flow of interstitial fluid sucked off from the organ by recording the change in conductance induced by the fluid flow. By monitoring the flow of fluid sucked off from the organ the treatment of the organ may be controlled. The applied negative pressure may be regulated in response to recorded changes in the fluid flow. For example, when the fluid flow falls to a level of the fluid flow from a healthy organ, an indication is given that no further treatment is needed at that moment.

Further, a metal contact surface may be used for applying a current to the body organ. The electrodes 13 used for detection may also be used for this purpose, or alternatively separate metal contacts are arranged for this purpose. The metal contact surface may in this case be arranged as a net layer of metal, which also may form the organ contacting surface 8. This enables application of a current to the body organ over a distributed area. Alternatively, the metal contact surface may be arranged as a metal wire around parts or the whole organ contacting surface 8.

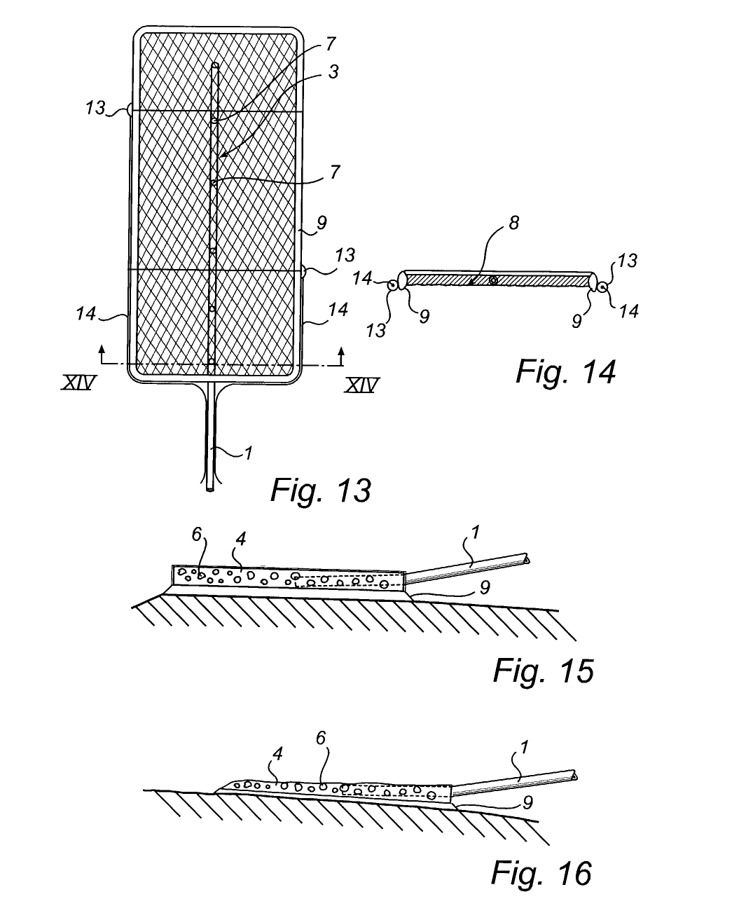

FIGS. 13-14 illustrate a variant of the device which substantially corresponds to the embodiment illustrated in FIGS. 1-3. However, the sealing ring 9 consists of a more rigid but still flexible ring and the chamber 8 has a rectangular shape instead of the oval shape shown in FIGS. 1-3. As in the embodiment of FIGS. 1-3, the tube 1 has a plurality of openings 7 in the distal end portion 3, and as in the variant shown in FIGS. 11-12, there are two electrodes 13 and two wires 14.

FIG. 15 illustrates a device according to the invention positioned on the tissue surface of a body organ with no suction applied. FIG. 16 illustrates the device in FIG. 15 with suction applied. The reduced pressure in the chamber 4 will compress the chamber 4 and give a suction effect on the tissue surface. As may be seen from FIG. 16, the compression of the chamber will not shrink the external surface of the chamber 4 in contact with the body organ in the plane of the surface. Thus, the part of the body organ in contact with the chamber 4 will not be immobilized by the suction.

FIG. 17 specifically illustrates the use of the device on a heart. In this application the device could be used for reconditioning a heart which e.g. suffers from ischemic areas caused by a myocardial infarction or from post cardiotomy syndrome.

In FIGS. 18-19 four devices according to FIGS. 13-14 are used for reconditioning of the lungs. FIG. 18 is a front view and FIG. 19 is a cross-sectional view along the lines XIX-XIX. Such application of the device could advantageously be used for treatment of e.g. ARDS, where excessive fluids is collected in the lungs.

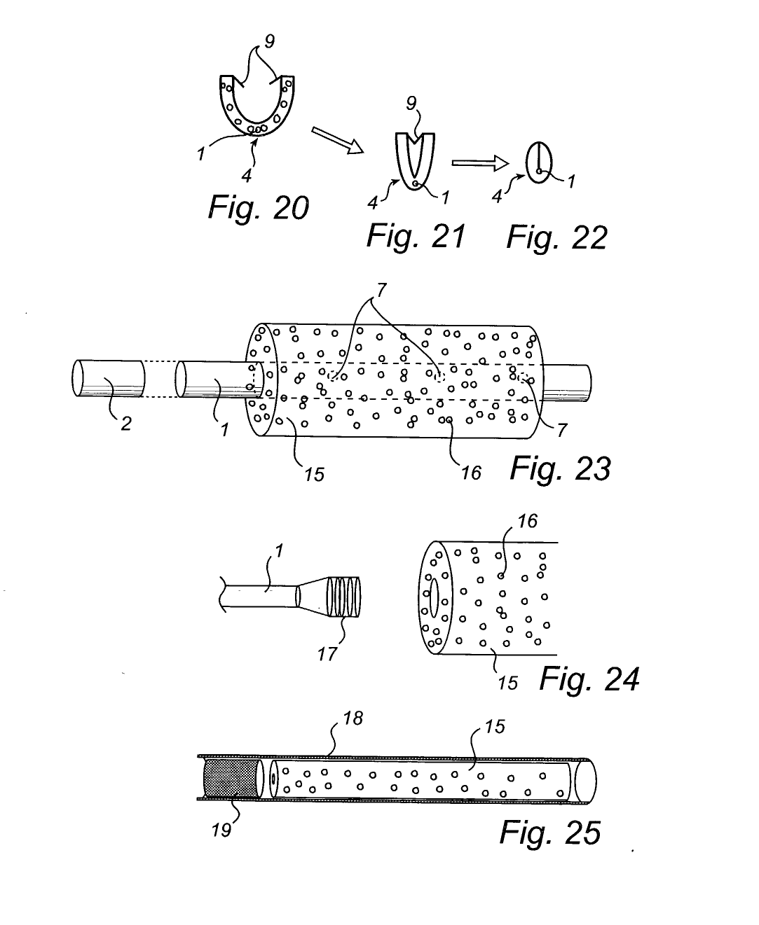

FIGS. 20-22 are cross-sectional views of the device shown in FIGS. 1-3. In FIG. 20 the device is folded along a centre line so that the sealing lips along opposite edges of the chamber 4 get in contact with each other, as shown in FIG. 21. Then the suction from the vacuum source is applied via the tube 1, whereby the device will shrink further to a minimal size, as illustrated in FIG. 22, permitting easy insertion into and through a cannula or tube. The cannula could then be used for introduction of the device into the body in a compressed state. The device may alternatively be rolled to form a cylindrical shape for insertion into the cannula.

As shown in FIG. 33, the device may alternatively be rolled around a guide wire 33 or a tube for insertion. Suction from the vacuum source may shrink the device to a small size around the guide wire 33. When the device has been inserted to the desired position within the body, it may be rolled off the guide wire 33 or tube and subsequently be used for sucking off fluids from the body organ.

An intraparenchymal device for insertion into the tissue of an organ is illustrated in FIGS. 23-25. This device has the same tube 1 as the device shown in FIGS. 13, but its chamber 15 is substantially cylindrical and totally occupied by a flexible material 16. More precisely, the chamber 15 may be defined by the peripheral surface of the flexible material 16 itself or may comprise a film which is perforated across a selected part of its external surface. Preferably, the selected part of the external surface of the chamber 15 includes the total peripheral part of the chamber 15.

The tube 1 may extend through the chamber 15, in which case the distal end of the tube 1 preferably is closed. Further, the tube 1 may have a permanent fixation to the flexible material 16 or it may be detachable by means of a quick connection coupling 17, as illustrated in FIG. 24. In this case, the flexible material 16 preferably is a resorbable material.

The flexible material 16 may be compressed so as to fit inside a cannula 18 for the delivery of the device. This delivery can be done by means of a piston 19, as illustrated in FIG. 25.

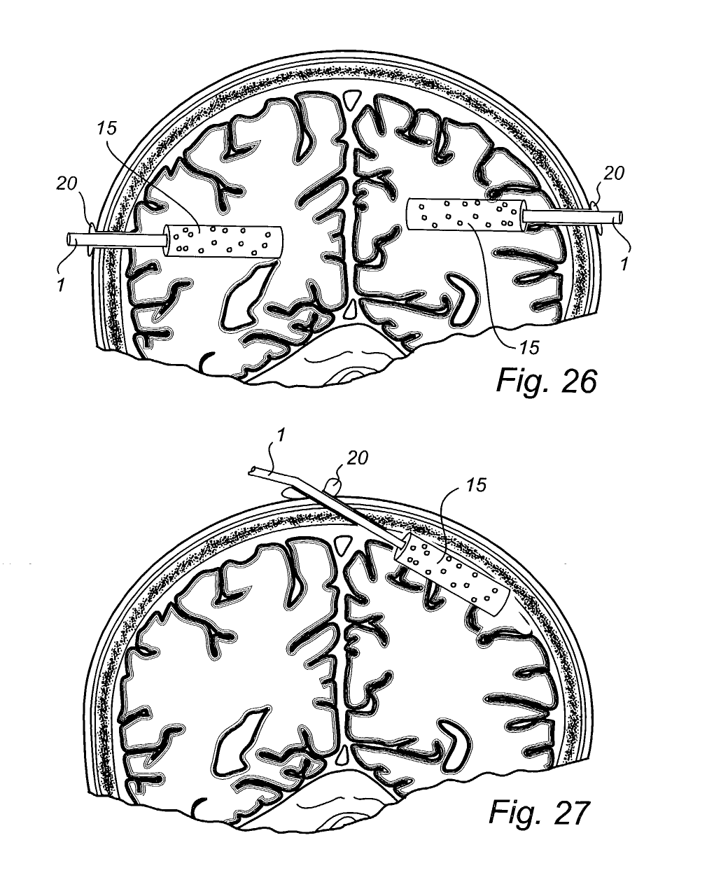

In FIG. 26, the intraparenchymal device according to FIG. 23 is inserted into the brain tissue. A special screw 20 fitting exactly to the suction tube 1 guarantees a tight sealing to the skull bone. In FIG. 27, the intraparenchymal device according to FIG. 23 is inserted under the skull bone either inside or outside the dura mater.

The device according to the present invention can be inserted directly or percutaneously by means of punction. Direct placement is done in the cases where direct access to the body organ in question is possible. Such direct placement would be possible in cases of open surgery where the surface of the body organ is exposed. Especially important is such direct placement during heart surgery and brain surgery when organs start to swell. Another situation when direct placement is possible is during transplantation of organs, after harvesting.

Special versions of the device are available for insertion and placement directly through the skin either by puncture or small incisions. The suction part of the device, i.e. the holes 7 of the distal end portion 3 of the tube 1 and the chamber 4 surrounding them, may then be compressed in different ways around the tube to make it as small as possible. One way to make the suction part small is to cover that part of the device with a film that is retractable, and then apply suction, whereby the device will be extremely slim and small.

Thus, the intraparenchymal device permits treatment of organs from the inside tissue of the organs rather than from the external surface thereof. When the device is implanted into the tissue, a film around the web is in this case not necessary. The web or parts thereof will be retrieved, when the device is pulled out of the organ. If a detachable tube is used, the web material preferably is resorbable in the body organ.

A method for reconditioning of a body organ comprises insertion of a device, which is described above, into contact with the organ. The chamber 4 surrounding the distal end portion 3 of the device could be compressed and inserted into a cannula so that it may easily be inserted into the body by key-hole surgery, or by catheter technique. When brought to the body organ to be reconditioned, the device is released from the cannula and the chamber 4 may be brought into contact with a surface of the body organ. The selected part 8 of the external surface and the sealing ring 9 enclosing it will be brought in contact with the surface of the body organ. When a negative pressure is applied, interstitial fluids of the body organ will be sucked off from the organ through fluid connections in the flexible material 6 of the chamber 4, through the suction openings 7 of the tube 1 and through the tube 1 into a receptacle outside the body. Thus, the flow of excessive fluid from the body organ is increased and the body organ is reconditioned.

The fluid which is sucked off the body organ typically comprises electrolytes, such as salt and water. None or at least insignificant amounts of proteins or cells are removed from the body organ with the fluid flow.

The applied negative pressure may be varied over different compartments 10-12 of the chamber 4 for varying the sucking off of fluids between different areas of the body organ. Further, the applied negative pressure may be varied in time. This may increase the efficiency of sucking off fluids, and thereby increase the efficiency of the device. Thus, the a cyclic variation of the applied negative pressure may be used, e.g. with a period of 1-3 minutes. Also, a much faster variation of the applied negative pressure may be triggered by the detected ECG as described above. This ECG-controlled variation may be used in stead of or in addition to the 1-3 minutes variation.

The applied negative pressure used is within the range of negative pressure used in the medical area, i.e. from 0 to 300 mmHg. Preferably, the applied negative pressure is within the range 25-125 mmHg.

Further, the device may be arranged so that a positive pressure may also be applied to the surface of the body organ. Thus, intermittently a positive pressure may be applied for a short period in order to break any granulation tissue that has been formed. Thereby, adhesion of the device to the surface of the body organ may be avoided.

The formation of granulation tissue may also be avoided by at certain intervals replacing the device in contact with the body organ. When the device is to be replaced a positive pressure might be applied so that any formed granulation tissue is broken.

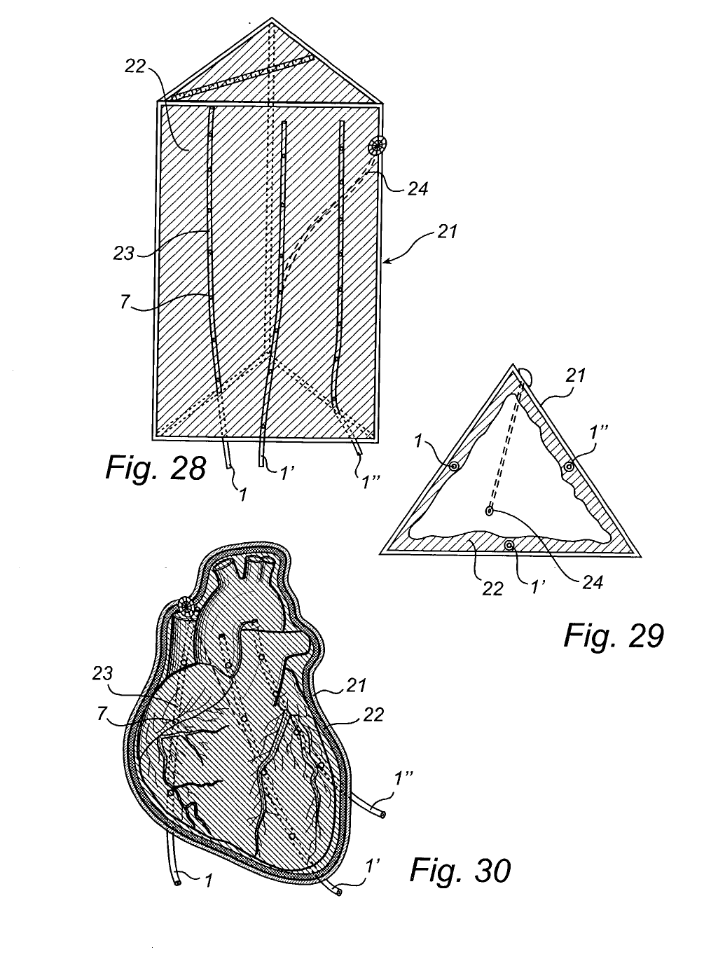

FIGS. 28-29 illustrate another embodiment of a device according to the present invention for carrying a body organ intended for transplantation. The device comprises a completely sealed soft synthetic bag 21 surrounding a web 22, into which several tubes 1, 1′, 1″ extend. The end portions 23 of the tubes 1, 1′, 1″ within the bag 21 has a plurality of openings 7 in the web 22, such that the atmosphere in the bag 21 may be evacuated by means of a vacuum source connected to the external ends of the tubes 1, 1′, 1″. As illustrated in FIG. 30, a heart placed in the bag 21 will be contacted by the web 22 on the inside of the bag 21 substantially all over its external surface, when the pressure in the bag 21 is decreased, whereby the preservation of the heart is improved during transport and storage thereof. During the transport the bag preferably is immersed in a cool transportation fluid or kept in a refrigerator or a cooling box. Further, a temperature probe 24 inside the bag permits constant monitoring of the organ temperature.

For the man skilled in the art it is obvious that the device may be modified in several aspects in order to be used for other organs, like the guts, the kidneys, the urinary tract and the liver. Also, the device may be brought in contact with both an internal and an external surface of the body organ simultaneously.

Further, the organ contacting surface need not be the only part of the device which is in contact with the body organ. The device may have other parts in contact with the body organ, through which no suction of interstitial fluids is created. The device may also be arranged such that the organ contacting surface is divided into separate parts, through all of which a suction of interstitial fluids may be created.

1. A device for reconditioning an internal body organ having or risking a functional failure associated with a fluid collection therein, said device comprising
a tube having a proximal end adapted for connection to a vacuum source, and a distal end portion having a plurality of openings,
a chamber surrounding the distal end portion of the tube and the openings therein, and
a flexible material occupying said chamber and forming fluid connections between a selected part of an external surface of the chamber and the openings of the distal end portion of the tube, said selected part of the external surface of the chamber being adapted for contacting the internal body organ,
whereby interstitial fluid of the internal body organ adjoining said selected part of the external surface of the chamber is sucked off from the internal body organ.

2. A device for reconditioning of an internal body organ having, or risking a functional failure or impairment associated with a fluid collection therein, said device comprising:
an organ contacting surface, which is adapted to contact the internal body organ, said organ contacting surface having, at least during the use of the device, pores allowing interstitial fluids to flow from the internal body organ through the surface, and
a draining element adapted to apply a negative pressure at the organ contacting surface and adapted to lead said interstitial fluids away from the internal body organ at said organ contacting surface via said pores.

3. A device as claimed in claim 2, wherein said pores of the organ contacting surface are initially clogged.

4. A device as claimed in claim 3, wherein the pores are clogged by a resorbable material.

5. A device as claimed in claim 2, wherein the organ contacting surface is adapted to contact an external surface portion of the body organ.

6. The device as claimed in claim 2, wherein the organ contacting surface has pores of a size sufficiently small to prevent stimulation of granulation tissue formation.

7. The device as claimed in claim 2, wherein the pores of the organ contacting surface are of a size in the interval 2-300 μm.

8. The device as claimed in claim 2, wherein the pores of the organ contacting surface are of a size in the interval 2-4 μm.

9. The device as claimed in claim 2, wherein the organ contacting surface has an essentially stable form.

10. The device as claimed in claim 9, wherein the organ contacting surface is pliable.

11. The device as claimed in claim 2, wherein the organ contacting surface restrains shrinking during suction.

12. The device as claimed in claim 2, wherein the organ contacting surface is covered by a perforated film.

13. The device as claimed in claim 2, wherein at least part of a peripheral surface of a porous body forms the organ contacting surface.

14. The device as claimed in claim 13, wherein the porous body is flexible.

15. The device as claimed in claim 13, wherein the porous body comprises a spongy material.

16. The device as claimed in claim 13, wherein the porous body comprises a web.

17. The device as claimed in claim 16, wherein the web comprises a synthetic tissue selected among the group consisting of polyester, polydiaxon, polyhexafluoropropylen-VDF, polyglyconat, polyglycolic acid, polyglactin, and silicon.

18. The device as claimed in claim 16, wherein the web comprises a metal material.

19. The device as claimed in claim 18, wherein the web comprises a Nitinol material.

20. The device as claimed in claim 16, wherein the web comprises a combination of a metal and a synthetic material.

21. The device as claimed in claim 16, wherein the web comprises at least one layer of a net.

22. The device as claimed in claim 21, wherein the net is fine-meshed.

23. The device as claimed in claim 13, wherein peripheral parts of the porous body not forming the organ contacting surface are covered by a film.

24. The device as claimed in claim 13, wherein a distal end of the draining element is arranged inside the porous body.

25. The device as claimed in claim 24, wherein the draining element comprises a tube.

26. The device as claimed in claim 25, wherein the distal end of the draining element comprises at least one suction opening in the tube.

27. The device as claimed in claim 2, wherein the draining element is connectable to a vacuum source.

28. The device as claimed in claim 2, further comprising a sealing ring, which encloses the organ contacting surface.

29. The device as claimed in claim 28, wherein the sealing ring is impermeable to fluids.

30. The device as claimed in claim 29, wherein the sealing ring is impermeable to gases as well.

31. The device as claimed in claim 28, further comprising means for creating a negative pressure between the sealing ring and a surface of the body organ, whereby the sealing ring may be fixated to the surface of the body organ.

32. The device as claimed in claim 28, wherein the sealing ring comprises a sealing lip.

33. The device as claimed in claim 13, wherein the porous body is divided into compartments, part of a peripheral surface of each compartment forming separate portions of the organ contacting surface.

34. The device as claimed in claim 33, wherein the draining element has a separate lumen for each compartment.

35. The device as claimed in claim 33, wherein the draining element comprises several tubes, wherein each tube comprises a distal end arranged in one of the compartments.

36. The device as claimed in claim 33, wherein a sealing ring encloses each portion of the organ contacting surface.

37. The device as claimed in claim 36, further comprising means for applying a negative pressure to each sealing ring.

38. The device as claimed in claim 36, wherein each sealing ring comprises a sealing lip.

39. A device as claimed in claim 13, wherein the organ contacting surface is adapted to contact an interior portion of the body organ.

40. The device as claimed in claim 13, wherein the porous body is substantially cylindrical and at least part of the peripheral surface of the substantially cylindrical porous body forms the organ contacting surface.

41. The device as claimed in claim 40, wherein the porous body comprises a resorbable material.

42. The device as claimed in claim 40, wherein the device further comprises a delivering cannula, into which the porous body is inserted in a compressed state.

43. The device as claimed in claim 2, further comprising an electrical current conducting means adapted for contacting the body organ.

44. A device as claimed in claim 43, wherein the electrical current conducting means comprises at least one electrode.

45. A device as claimed in claim 43, wherein the electrical current conducting means is adapted for detecting an electrical current.

46. A device as claimed in claim 45, wherein the electrical current conducting means is adapted for detecting an electrical current in the body organ.

47. A device as claimed in claim 45, wherein the electrical current conducting means is adapted for detecting an electrical current influenced by the fluid flow away from the body organ.

48. A device as claimed in claim 43, wherein the electrical current conducting means is adapted for applying an electrical current to the body organ.

49. A device as claimed in claim 48, wherein the electrical current conducting means comprises a net layer of metal for applying an electrical current to the body organ over a distributed surface.

50. A system for reconditioning an internal body organ having, or risking a functional failure or impairment associated with a fluid collection therein, said system comprising
an organ contacting device, said organ contacting device including
an organ contacting surface, which is adapted to contact the internal body organ, said organ contacting surface having pores allowing interstitial fluids to flow through the surface, and
a draining element adapted to apply a negative pressure at the organ contacting surface and adapted to lead sucked off fluids away from the internal body organ at said organ contacting surface, and
a negative pressure source for connection to the draining element and for creation of the negative pressure at the organ contacting surface.

51. A method for reconditioning an internal body organ having or risking a functional failure associated with a fluid collection therein, comprising the steps of
providing a tube, which has a proximal end and a distal end portion having a plurality of openings, a chamber surrounding the distal end portion of the tube and the openings therein, and a flexible material in said chamber and forming fluid connections to the openings of the distal end portion of the tube;
contacting the internal body organ by said flexible material; and
connecting the proximal end of the tube to a vacuum source,
whereby interstitial fluid of the internal body organ adjoining the flexible material is sucked off from the internal body organ.

52. A method as claimed in claim 51, wherein the suction provided by the vacuum source is cyclically varied.

53. A method as claimed in claim 52, wherein the cyclical variation has an interval of 1-3 minutes.

54. A method as claimed in 52 for reconditioning of a heart, wherein the suction is varied in response to the electrocardiogram.

55. A method as claimed in claim 51 for curing an unstable angina pectoris.

56. A method as claimed in claim 51 for increasing the blood flow in the treated body organ.

57. A method as claimed in claim 51, wherein the applied suction corresponds to a pressure of 25-125 mmHg.

58. A method as claimed in claim 51, further comprising the step of prohibiting, or at least substantially reducing, stimulation of granulation tissue at a contact area of the flexible material to the surface of the body organ.

59. A method as claimed in claim 51, further comprising the step of prohibiting, or at least substantially reducing, stimulation of adhesion of the contact area to the surface of the body organ.

60. A method as claimed in claim 51, further comprising the step of monitoring the flow of interstitial fluid sucked off from the body organ.

61. A method as claimed in claim 60, further comprising the step of regulating a degree of vacuum in the vacuum source in dependence of the monitored flow.

62. A method as claimed in claim 60, further comprising the step of shutting off the vacuum source when the monitored flow is at a normal level.

63. A method as claimed in claim 51, further comprising the step of detecting an electrical current in the body organ.

64. A method for reconditioning of an internal body organ having, or risking a functional failure or impairment associated with a fluid collection therein, said method comprising the steps of:
contacting the internal body organ with a suction device, and
creating a negative pressure in a contact area between the internal body organ and the suction device,
whereby interstitial fluid is sucked off from the internal body organ.

65. A method for reconditioning of an internal body organ having, or risking a functional failure or impairment associated with a fluid collection therein, said method comprising the step of:
sucking off interstitial fluid from an external surface or from the interior of the internal body organ.

66. A method for reconditioning of an internal body organ having, or risking a functional failure or impairment associated with a fluid collection therein, said method comprising the step of:
increasing interstitial fluid flow away from the internal body organ by applying a negative pressure to the internal body organ.

67. A method as claimed in claim 66, further comprising the step of applying a negative pressure to an external surface of the body organ.

68. A method as claimed in claim 66, further comprising the step of applying a negative pressure to the interior of the body organ.

69. A device for preserving a body organ for transport purposes, said device comprising
having a web inside,
at least one tube having a proximal end adapted for connection to a vacuum source, and a distal end portion having a plurality of openings,
a flexible sealed bag surrounding the distal end portion of the tube and the openings therein, and
a flexible material positioned on the inside of the flexible bag, the openings in the distal end of the tube being positioned within the web, which forms fluid connections between an external surface of a body organ placed in the bag and the openings of the distal end portion of the tube,
whereby interstitial fluid of the body organ adjoining the web is sucked off from the body organ.

70. A device for preserving a body organ for transport purposes, said device comprising:
a sealable bag having an internal space for receiving a body organ, and
a draining element adapted to apply a negative pressure to an external surface of the body organ and adapted to lead sucked off fluids away from the body organ.

71. The device as claimed in claim 70, further comprising an organ contacting surface located within the bag and adapted to contact the body organ, said organ contacting surface being connected to the draining element for applying a negative pressure to the external surface of the body organ.

72. The device as claimed in claim 71, wherein the organ contacting surface is formed by a peripheral surface of a porous body.

73. The device as claimed in claim 72, wherein the porous body comprises a flexible material.

74. The device as claimed in claim 72, wherein the porous body comprises a web.

75. The device as claimed in claim 70, wherein the draining element comprises at least one tube.

76. The device as claimed in claim 75, wherein at least one suction opening is arranged at a distal end of each tube.

77. The device as claimed in claim 76, wherein the distal end of each tube is arranged in the porous body.

78. The device as claimed in claim 70, wherein the draining element is adapted for connection to a vacuum source.
